(19) United States
(12) Patent Application Publication
(10) Pub. No.: US 2018/0333138 A1
(43) Pub. Date: Nov. 22, 2018

(54) ULTRASONIC DIAGNOSTIC APPARATUS, AND ULTRASONIC DIAGNOSTIC METHOD

(71) Applicant: Canon Medical Systems Corporation, Otawara-shi (JP)

(72) Inventors: Takatoshi OKUMURA, Yaita (JP); Kazutoshi SADAMITSU, Otawara (JP); Yuhei FUKUO, Nasushiobara (JP); Norihisa KIKUCHI, Otawara (JP); Masatoshi NISHINO, Otawara (JP); Yoshinori GOTO, Nasushiobara (JP); Naoki YONEYAMA, Yaita (JP)

(73) Assignee: Canon Medical Systems Corporation, Otawara-shi (JP)

(21) Appl. No.: 15/980,173

(22) Filed: May 15, 2018

(30) Foreign Application Priority Data
May 16, 2017 (JP) 2017-097110

(51) Int. Cl. A61B 8/00; A61B 8/08

(52) U.S. Cl. CPC A61B 8/463; A61B 8/54; A61B 8/4254; A61B 2562/0247; A61B 8/085

(57) ABSTRACT

According to one embodiment, an ultrasonic diagnostic apparatus includes a processor. The processor generates assistance information to assist an operation of a puncture needle based on pressure information acquired from a pressure sensor attached to the puncture needle. The processor displays on a display the assistance information and an ultrasonic image which includes at least an image of a tip of the puncture needle, the ultrasonic image being acquired by transmission and reception of ultrasonic waves by using an ultrasonic probe.

### CROSS-REFERENCE TO RELATED APPLICATIONS

This application is based upon and claims the benefit of priority from the prior Japanese Patent Application No. 2017-097110, filed May 16, 2017, the entire contents of which are incorporated herein by reference.

### FIELD

Embodiments described herein relate generally to an ultrasonic diagnostic apparatus, and an ultrasonic diagnostic method.

### BACKGROUND

A method of performing a predetermined inspection or diagnosis by inserting a puncture needle into a region of interest (ROI) such as a tumor of a patient (an inspection/diagnosis target) while observing an image obtained by an ultrasonic diagnostic apparatus has been developed. For example, while a two-dimensional image acquired in cross sections that include a puncture needle is displayed, an operator observes a tumor and the puncture needle, and inserts the puncture needle into the tumor based on the displayed positional relationships between them. For the purpose of assisting accurate insertion of a puncture needle, a puncture guide is superimposed to the two-dimensional image. The puncture guide is an indication of an expected insertion path of a puncture needle.

It is premised that a puncture needle is linearly inserted into a body of a patient. However, a general puncture needle does not have sufficient hardness. Thus, if elasticity (hardness) of a vital tissue on the insertion path is nonuniform, there may be a case where the puncture needle is inserted in a direction deviated from the expected insertion path indicated by the puncture guide.

To address this problem, there are methods for displaying a position of a puncture needle in which a tip of the puncture needle is displayed based on a three-dimensional image data (volume data), or for displaying an indicator of an estimated position of the puncture needle corrected based on a difference between the indicator and an actual position of the puncture needle on a two-dimensional image.

An operator needs to correct an insertion direction of a needle tip by predicting a deviation from the puncture guide so that the puncture needle is inserted along the puncture guide. However, if the insertion direction of the puncture needle is deviated from the puncture guide, it is difficult to determine how to correct the insertion direction based only on recognition of the tip of the puncture needle or an image showing the actual position of the puncture needle. Accordingly, the correction of the insertion direction of the puncture needle may rely on the operator's expertise or experiences. As a result, there is a problem that the puncturing accuracy varies.

### BRIEF DESCRIPTION OF THE DRAWINGS

FIG. 1 is a block diagram showing the configuration of an ultrasonic diagnostic apparatus according to a first embodiment.

FIG. 2A illustrates the first example of a pressure sensor attached to a puncture needle.

FIG. 2B illustrates the second example of a pressure sensor attached to a puncture needle.

FIG. 3 illustrates the third example of a pressure sensor attached to a puncture needle.

FIG. 4 illustrates an operation principle of pressure detection by an optical fiber sensor.

FIG. 5 is a flowchart of an operation of the ultrasonic diagnostic apparatus.

FIG. 6 illustrates the first display example of assistance information.

FIG. 7 illustrates the second display example of assistance information.

FIG. 8 illustrates the third display example of assistance information.

FIG. 9 illustrates a display example of a needle tip end position of a puncture needle.

FIG. 10 illustrates the fourth display example of assistance information.

FIG. 11 illustrates a display control example of assistance information by a display control function.

FIG. 12 illustrates a display example of an expected insertion line.

FIG. 13 illustrates a case where correction information is displayed as assistance information.

FIG. 14 illustrates a result where a puncture needle is rotated by the amount indicated by correction information.

### DETAILED DESCRIPTION

In general, according to one embodiment, an ultrasonic diagnostic apparatus includes a processor. The processor generates assistance information to assist an operation of a puncture needle based on pressure information acquired from a pressure sensor attached to the puncture needle. The processor displays on a display the assistance information and an ultrasonic image which includes at least an image of a tip of the puncture needle, the ultrasonic image being acquired by transmission and reception of ultrasonic waves by using an ultrasonic probe.

In the following descriptions, an ultrasonic diagnostic apparatus and an ultrasonic diagnosis method according to the present embodiments will be described with reference to the drawings. In the embodiments described below, elements assigned with the same reference symbols perform the same operations, and redundant descriptions thereof will be omitted as appropriate.

### First Embodiment

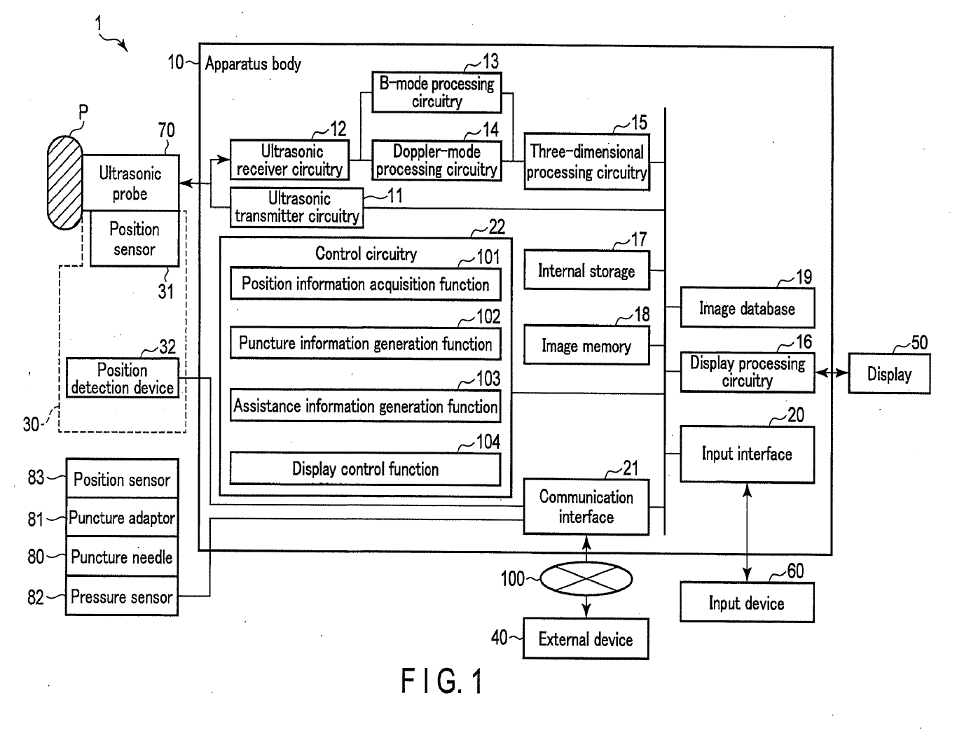

An ultrasonic diagnostic apparatus according to the first embodiment will be described with reference to the block diagram shown in FIG. 1.

As shown in FIG. 1, an ultrasonic diagnostic apparatus 1 includes an apparatus body 10, an ultrasonic probe 70, and a position sensor system 30. The apparatus body 10 is connected to an external device 40 through a network 100. The apparatus body 10 is connected to a display 50 and an input device 60. The ultrasonic diagnostic apparatus 1 according to the first embodiment is assumed to be used in paracentesis, and the ultrasonic probe 70 and a puncture needle 80 are used together.

The position sensor system 30 is configured to acquire three-dimensional positional information of the ultrasonic probe 70 and an ultrasonic image. The position sensor system 30 includes a position sensor 31 and a position detection device 32.

The position sensor system 30 acquires three-dimensional positional information of the ultrasonic probe 70 by using a magnetic sensor, an infrared sensor, a target for an infrared camera, etc. attached to the ultrasonic probe 70 as the position sensor 31. A gyro sensor (angular velocity sensor) may be installed in the ultrasonic probe 70 so that the gyro sensor acquires the three-dimensional positional information of the ultrasonic probe 70. The position sensor system 30 may acquire the three-dimensional positional information of the ultrasonic probe 70 by imaging the ultrasonic probe 70 by a camera and performing image recognition processing to the image of the ultrasonic probe 70. The position sensor system 30 may detect a three-dimensional position of a robot arm that holds the ultrasonic probe 70 and acquires the position of the robot arm as positional information of the ultrasonic probe 70.

An example where the position sensor system 30 acquires the positional information of the ultrasonic probe 70 by using the magnetic sensor will be described below. Specifically, the position sensor system 30 further includes, for example, a magnetic generator (not illustrated) having a magnetic generation coil, etc. The magnetic generator forms a magnetic field oriented outward from the magnetic generator itself. A magnetic space in which location accuracy is assured is defined in the formed magnetic field. The magnetic generator may be arranged so that a living body to be examined is placed within the magnetic space in which location accuracy is assured. The position sensor 31 attached to the ultrasonic probe 70 detects the strength and gradient of a three-dimensional magnetic field formed by the magnetic generator. By this detection, a position and an orientation of the ultrasonic probe 70 can be acquired. The position sensor 31 outputs the detected strength and inclination of the magnetic field to the position detection device 32.

The position detection device 32 calculates, for example, a position (a position on a scanning plane (x, y, z) and an rotation angle (θx, θy, θz)) of the ultrasonic probe 70 in the three-dimensional space in which a predetermined position is set to an origin, based on the strength and gradient of the magnetic field detected by the position sensor 31. The predetermined position is assumed to be, for example, a position where the magnetic generator is placed. The position detection device 32 transmits to the apparatus body 10 positional information relating to the calculated position (x, y, z, θx, θy, θz).

By associating the acquired positional information with ultrasonic image data obtained by ultrasonic waves transmitted and received relative to the ultrasonic probe 70 by time synchronization, etc., the positional information can be added to the ultrasonic image data.

The ultrasonic probe 70 includes a plurality of piezoelectric transducers, a matching layer provided to the piezoelectric transducers, and a backing material that prevents propagation of ultrasonic waves to the rear side of the piezoelectric transducers, etc. The ultrasonic probe 70 is detachably connected to the apparatus body 10. The piezoelectric transducers generate ultrasonic waves based on a driving signal supplied from the ultrasonic transmitter circuitry 11 of the apparatus body 10. The ultrasonic probe 70 may be provided with a button used for offset processing or in case of freezing of an ultrasonic image, described later.

Once the ultrasonic probe 70 transmits ultrasonic waves to a living body P, the transmitted ultrasonic waves are sequentially reflected by the boundary showing discontinuity of the acoustic impedance of the living tissue of the living body P, and are received by the plurality of piezoelectric transducers of the ultrasonic probe 70 as reflected wave signals. The amplitude of the received reflected wave signals depends on the difference in the acoustic impedance at the boundary showing discontinuity of the acoustic impedance that affects the reflection of ultrasonic waves. If the transmitted ultrasonic pulses are reflected in a bloodstream or on the surface of the cardiac wall, the frequency of the reflected wave signals is shifted depending on velocity components in the direction of transmitting ultrasonic waves in a moving object due to the Doppler effect. The ultrasonic probe 70 receives the reflected wave signals from the living body P, and converts the reflected wave signals into electrical signals.

As stated above, since the ultrasonic probe 70 according to the present embodiment is provided with the position sensor 31, the ultrasonic probe 70 can detect positional information when performing three-dimensional scanning on the living body P. The ultrasonic probe 70 according to the present embodiment is assumed to be able to generate volume data. For example, the ultrasonic probe 70 may be a two-dimensional array type probe in which a plurality of ultrasonic transducers are arranged in a form of a matrix. Alternatively, the ultrasonic probe 70 may be a mechanical four-dimensional probe (mechanical 3D dynamic probe) that includes a one-dimensional array probe and a probe sweeping motor in an enclosure, and mechanically performs a sweep scan or a rotating scan by sweeping the ultrasonic transducers in a predetermined angle (fluctuation angle), and three-dimensionally scans the living body P. In addition, the ultrasonic probe 70 may be a 1.5D array probe in which one-dimensionally arranged transducers are divided into groups.

The processing of the present embodiment is applicable not only to volume data, but also to the two-dimensional ultrasonic image acquired by a scan in the cross sections including the puncture needle 80. Accordingly, the ultrasonic probe 70 may be a one-dimensional array probe in which a plurality of ultrasonic transducers are simply arranged in a line in the array direction.

The ultrasonic probe 70 is provided with a puncture adaptor 81. The puncture adaptor 81 is capable of defining a puncture initial position of the puncture needle 80, and of holding the puncture needle 80 slidably relative to the insertion direction. The puncture adaptor 81 can discretionarily adjust the puncture angle of the puncture needle 80. The puncture needle 80 is inserted into a living body along a scanning surface of ultrasonic waves by using the puncture adaptor 81. The ultrasonic probe 70 and the puncture adaptor 81 may be separated from each other, and the ultrasonic probe 70 and the puncture needle 80 may be discretionarily operated independently without using the puncture adaptor 81.

The puncture needle 80 can be any type of puncture needle. For example, the puncture needle 80 may be a puncture needle for biopsy for the purpose of sampling of a tumor tissue, or a puncture needle for cauterization such as an RFA puncture needle capable of cauterizing a tumor.

The puncture needle 80 is provided with a pressure sensor 82 and a position sensor 83. The pressure sensor 82 measures a degree of pressure (a pressure value, for example) applied to the puncture needle 80 at a plurality of positions on the side surface. The position sensor 83 is different from the position sensor 31 provided in the ultrasonic probe 70. The position sensor 83 measures at least the position of a tip of the puncture needle 80 to obtain positional information of the puncture needle 80. The position sensor 83 may transmit a radio signal to an external receiver, and the receiver may calculate positional information of the puncture needle 80 based on the radio signal. The position sensor 83 may be a generally used sensor that measures a position of the puncture needle 80. Accordingly, the detailed explanation is omitted.

The apparatus body 10 shown in FIG. 1 generates an ultrasonic image, based on reflected wave signals received by the ultrasonic probe 70. As shown in FIG. 1, the apparatus body 10 includes ultrasonic transmitter circuitry 11, ultrasonic receiver circuitry 12, B-mode processing circuitry 13, Doppler-mode processing circuitry 14, three-dimensional processing circuitry 15, display processing circuitry 16, an internal storage 17, an image memory 18 (cine memory), an image database 19, input interface 20, communication interface 21, and control circuitry 22.

The ultrasonic transmitter circuitry 11 is a processor that supplies driving signals to the ultrasonic probe 70. The ultrasonic transmitter circuitry 11 is implemented, for example, by trigger generating circuitry, delay circuitry, and pulser circuitry, etc. The trigger generating circuitry repeatedly generates rate pulses for forming transmission ultrasonic waves at a predetermined rate frequency. The delay circuitry converges ultrasonic waves generated from the ultrasonic probe 70 as a beam, and applies, to each rate pulse generated by the trigger generating circuitry, a transmission delay time for each ultrasonic transducer required for determining a transmission directivity. The pulser circuitry supplies driving signals (driving pulses) to the ultrasonic probe 70 at a timing based on the rate pulse. By changing the transmission delay time to be applied to each rate pulse from the delay circuitry, the transmission direction from the ultrasonic transducer surface can be discretionarily adjusted.

The ultrasonic receiver circuitry 12 is a processor that executes various processes on reflected wave signals received by the ultrasonic probe 70 to generate a receive signal. The ultrasonic receiver circuitry 12 is implemented, for example, by amplification circuitry, an A/D converter, reception delay circuitry, and an adder, etc. The amplification circuitry executes a gain correction processing for each channel by amplifying reflected wave signals received by the ultrasonic probe 70. The A/D converter converts the gain-corrected reflected wave signals to digital signals. The reception delay circuitry delays input of the digital signals to an adder by a reception delay time required for determining a reception directivity. The adder adds a plurality of digital signals in which the reception delay time has been applied. After the addition processing of the adder, receive signals are generated in which a reflected component from a direction corresponding to the reception directivity is emphasized.

The B-mode processing circuitry 13 is a processor that generates B-mode data, based on the receive signals received from the ultrasonic receiver circuitry 12. The B-mode processing circuitry 13 executes an envelope detection process and a logarithmic amplification process, etc. on the receive signals received from the ultrasonic receiver circuitry 12, and generates data (B-mode data) in which the signal intensity is expressed by the brightness intensity. The generated B-mode data is stored in a not-illustrated RAW data memory as B-mode RAW data on a two-dimensional ultrasonic scanning line.

The Doppler-mode processing circuitry 14 is a processor that generates a Doppler waveform and Doppler data, based on the receive signals received from the ultrasonic receiver circuitry 12. The Doppler-mode processing circuitry 14 extracts a blood flow signal from the receive signal, generates a Doppler waveform from the extracted blood flow signal, and generates data (Doppler data) in which information, such as a mean velocity, dispersion, power, etc. is extracted from the blood flow signal with respect to multiple points.

The three-dimensional processing circuitry 15 is a processor that can generate volume data along with positional information (three-dimensional image data along with positional information), based on the data generated by the B-mode processing circuitry 13 and the Doppler-mode processing circuitry 14.

In addition, the three-dimensional processing circuitry 15 performs, to B-mode RAW data stored in the RAW data memory, RAW-voxel conversion which includes an interpolation process taking spatial position information into consideration to generate volume data consisting of voxels in a desired range. In the case where the ultrasonic probe 70 provided with the position sensor 31 is a mechanical 4D probe (mechanical 3D dynamic probe), or a 2D array probe, positional information calculated by the position detection device 32 is added to volume data.

Similarly, in the case where the ultrasonic probe 70 provided with the position sensor 31 is a 1D array probe, the three-dimensional processing circuitry 15 adds positional information of the ultrasonic probe 70 calculated by the position detection device 32 to the B-mode RAW data stored in the RAW data memory. Specifically, the three-dimensional processing circuitry 15 executes RAW-voxel conversion to generate two-dimensional image data consisting of pixels, and adds positional information of the ultrasonic probe 70 calculated by the position detection device 32 to the generated two-dimensional image data.

The three-dimensional processing circuitry 15 generates rendering image data by performing a rendering process to the generated volume data.

The display processing circuitry 16 executes various processes, such as dynamic range, brightness, contrast and γ curve corrections, and RGB conversion, etc. to image data generated in the three-dimensional processing circuitry 15, in order to convert the image data to video signals. The display processing circuitry 16 directs the display 50 to display an ultrasonic image in accordance with the video signal. The display processing circuitry 16 may generate a user interface (GUI: Graphical User Interface) through which an operator (for example, a surgeon) inputs various instructions by the input interface 20, and may direct the display 50 to display the GUI. The display 50 may adopt, for example, a CRT display, a liquid crystal display, an organic EL display, an LED display, a plasma display, or any other displays known in this technical field.

The internal storage 17 includes, for example, a storage medium which is readable by a processor, such as a magnetic or optical storage medium, or a semiconductor memory, etc. The internal storage 17 stores a control program for implementing ultrasonic transmission/reception, a control program for executing an image process, and a control program for executing a display process, etc. In addition, the internal storage 17 stores diagnosis information (e.g., patient ID, doctor's findings, etc.), a diagnosis protocol, a body mark generation program, and data such as a conversion table for presetting a range of color data for use in visualizing, with respect to each of regions of diagnosis. The internal storage 17 may store anatomical illustrations, for example, an atlas, relating to the structures of internal organs in the body.

In addition, the internal storage 17 stores two-dimensional image data, volume data, and rendering image data generated by the three-dimensional processing circuitry 15, in accordance with a storing operation input through the input interface 20. In accordance with the storing operation input through the input interface 20, the internal storage 17 may store volume data along with positional information, rendering image data along with positional information, and two-dimensional image data along with positional information, generated by the three-dimensional processing circuitry 15, together with an operation order and an operation time. The internal storage 17 can transfer the stored data to an external device through the communication interface 21.

The image memory 18 includes, for example, a storage medium which is readable by a processor, such as a magnetic or optical storage medium, or a semiconductor memory. The image memory 18 stores image data corresponding to a plurality of frames immediately before a freeze operation input through the input interface 20. The image data stored in the image memory 18 is successively displayed (cine-displayed), for example.

The image database 19 stores image data transferred from the external device 40. For example, the image database 19 acquires and stores past image data relating to a particular patient obtained by the past diagnosis and stored in the external device 40. The past image data includes ultrasonic image data, computed tomography (CT) image data, MR image data, positron emission tomography (PET)-CT image data, PET-MR image data, and X-ray image data.

The image database 19 may store desired image data by reading image data stored in a storage medium such as an MO, a CD-R, and a DVD.

The input interface 20 receives various instructions from an operator through the input device 60. The input device 60 is, for example, a mouse, a keyboard, a panel switch, a slider switch, a trackball, a rotary encoder, an operation panel, and a touch command screen (TCS). The input interface 20 is connected to the control circuitry 22, for example, via a bus. The input interface 20 converts an operation instruction input by the operator into electric signals, and outputs the electric signals to the control circuitry 22. In the present embodiments, the input interface 20 is not limited to be connected to physical operation components such as a mouse, a keyboard, etc. The input interface 20 may include processing circuitry of electric signals which receives, as radio signals, electric signals corresponding to an operation instruction input from an external input device independently provided from the ultrasonic diagnostic apparatus 1, and outputs the electric signals to the control circuitry 22.

The communication interface 21 is, for example, wirelessly connected to the position sensor system 30, and receives positional information transmitted from the position detection device 32. The communication interface 21 is connected to the external device 40 through the network 100, etc., and performs data communication with the external device 40. The external device 40 is, for example, a database of a picture archiving and communication system (PACS) which is a system for managing various medical image data, a database of an electronic medical record system for managing electronic medical records along with medical images, etc. In addition, the external device 40 may, for example, be any medical image diagnostic apparatus other than the ultrasonic diagnostic apparatus 1 according to the present embodiment, such as an X-ray CT apparatus, an MRI (Magnetic Resonance Imaging) apparatus, a nuclear medical diagnostic apparatus, and an X-ray diagnostic apparatus, etc. Any standards may be applied for communication with the external device 40. For example, digital imaging and communication in medicine (DICOM) may be applied.

The control circuitry 22 is a processor acting as a nerve center of the ultrasonic diagnostic apparatus 1, for example. The control circuitry 22 executes a control program stored in the internal storage to realize a function corresponding to the program. Specifically, the control circuitry 22 activates a position information acquisition function 101, a puncture information generation function 102, an assistance information generation function 103, and a display control function 104.

By activating the position information acquisition function 101, the control circuitry 22 acquires, through the communication interface 21, positional information relating to the ultrasonic probe 70 from the position sensor system 30, and positional information relating to the puncture needle 80 from the position sensor 83.

By activating the puncture information generation function 102, the control circuitry 22 generates puncture information relating to at least a position of a tip of the puncture needle 80 and an insertion direction of the puncture needle 80, based on the sequentially acquired positional information relating to the puncture needle 80, and the sequentially acquired positional information relating to the ultrasonic probe 70. By activating the puncture information generation function 102, the control circuitry 22 generates a puncture guide indicating an expected insertion path to a living body part (target part) such as a tumor to which the puncture needle 80 is to be inserted within a scan area.

By activating the assistance information generation function 103, the control circuitry 22 generates assistance information to assist an operation of the puncture needle 80 based on pressure information at a plurality of positions on the puncture needle side surface obtained by the pressure sensors 82.

By activating the display control function 104, the control circuitry 22 controls at least display of the ultrasonic image and the assistance information. For example, the display control function 104 may control the display 50 to display the ultrasonic image of a cross section which includes the puncture needle selected based on the puncture information, the puncture guide and the assistance information. The display control function 104 may output the ultrasonic image, the puncture guide, and the assistance information to the external device 40, in order for a display, etc. of the external device 40 connected through the network 100 to display them.

The position information acquisition function 101, the puncture information generation function 102, the assistance information generation function 103, and the display control function 104 may be installed as a control program, or may be installed as a hardware circuit specific to each function in the control circuitry 22 or in the apparatus body 10 in a manner that the control circuitry 22 can refer to the hardware circuit.

The control circuitry 22 may be implemented by a circuit or a device which includes a hardware circuit specific to these functions such as an application specific integrated circuit (ASIC), an field programmable gate array (FPGA), a complex programmable logic device (CPLD), or an simple programmable logic device (SPLD).

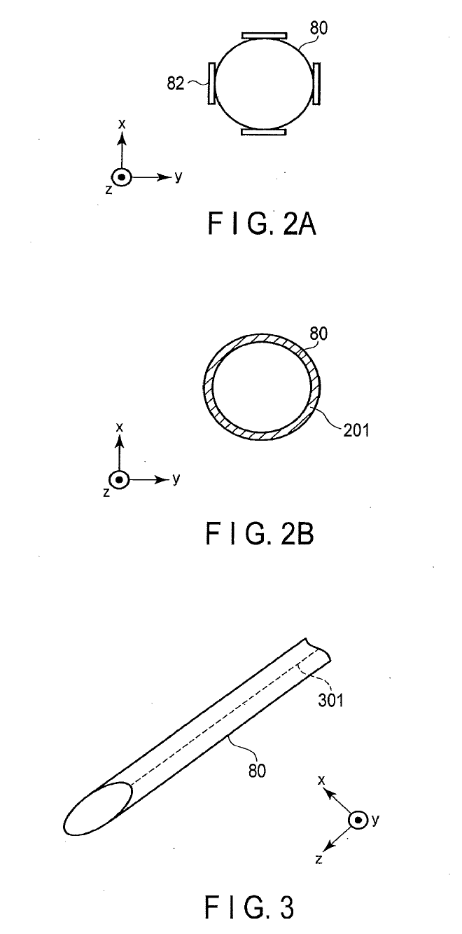

The first example of the pressure sensor 82 attached to the puncture needle 80 according to the present embodiment will be described with reference to FIG. 2A.

FIG. 2A is a cross-sectional view of the puncture needle 80 from the tip side. Four pressure sensors 82 are provided at the top, bottom, right-side and left-side of the puncture needle 80 in the cross section. With this arrangement, pressure values in four directions from the outside to the inside of the puncture needle can be detected. With the arrangement of the pressure sensors 82 as shown in FIG. 2A, the control circuitry 22 that activates the assistance information generation function 103 calculates a difference between the pressure values of two pressure sensors 82 facing each other, and calculates a degree of pressure applied to the puncture needle 80 and a direction of a mechanical load applied to the puncture needle 80. The calculation results indicate that the mechanical load is applied to the puncture needle 80 at the side of the pressure sensor 82 that shows a smaller pressure value. Accordingly, the operator can predict a direction where the puncture needle 80 is to be deviated from the puncture guide (a direction to which the puncture needle 80 may curve).

The pressure sensor 82 may adopt any sensors capable of measuring a distortion amount such as a piezoelectric element, etc. which is formed of a general semiconductor. The pressure sensors 82 are not limited to be arranged at four points on a circumference of the puncture needle 80. The number of the pressure sensors 82 may vary. For example, the pressure sensors 82 may be arranged at five or more points of a circumference, or at multiple points on a circumference at predetermined intervals. By adopting multiple pressure sensors 82, the direction of the mechanical load applied to the puncture needle 80 can be detected more precisely.

In addition, it is assumed that the pressure sensors 82 are arranged in a range at least within 3 cm from the tip end (insertion side) to the rear end (operator side) of the puncture needle 80. However, the pressure sensors 82 may be arranged over the entire puncture needle 80.

The second example of the pressure sensor 82 attached to the puncture needle 80 according to the present embodiment will be described with reference to FIG. 2B.

Instead of arranging the individual pressure sensors at multiple points, a distortion sensor 201 may be wound around the puncture needle 80 to form the pressure sensor 82. The distortion sensor 201 may be formed of a metallic thin film, etc. mounted to a surface of the puncture needle 80. By using the principle regarding an electric resistance distortion, the distortion sensor 201 can detect a potential difference from a resistance value at a time when a distortion (deflection, curvature) occurs, and can calculate a pressure value from the detected potential difference. With this structure, more specific information on the direction of the mechanical load applied to the puncture needle 80 can be acquired.

The distortion sensor formed of a thin film may be wound in a range at least within 3 cm from the tip end to the rear end of the puncture needle 80, or may be wound over the entire puncture needle 80, similar to the pressure sensors individually arranged at multiple points. If the distortion sensor is wound over the entire puncture needle 80, a degree of distortion of the entire puncture needle 80 (a degree of deviation of the entire puncture needle 80 from the puncture guide) can be detected, and this enables easier prediction of the direction of deviation from the puncture guide.

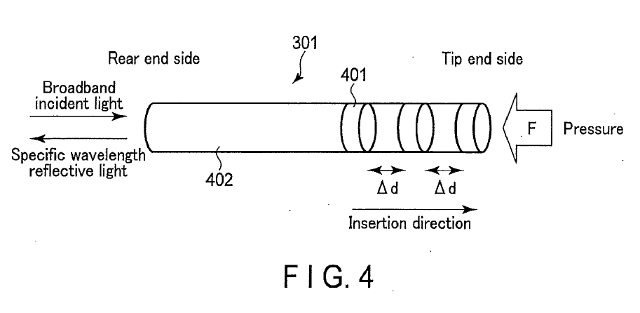

The third example of the pressure sensor 82 attached to the puncture needle 80 will be described with reference to FIGS. 3 and 4.

FIG. 3 shows an example of arrangement of an optical fiber sensor 301 which is used as the pressure sensor 82. As shown in FIG. 3, the optical fiber sensor 301 extends along a longitudinal direction of the puncture needle 80. It is desired that the optical fiber sensor 301 is arranged inside of the puncture needle 80, but the optical fiber sensor 301 may be provided to cover the puncture needle 80, or provided along the surface of the puncture needle 80. In the case where the optical fiber sensor 301 can detect both of a pressure value and a direction of pressure, one optical fiber sensor 301 may be sufficient. If the optical fiber sensor 301 can detect only a pressure value, at least four optical fiber sensors 301 are arranged around the puncture needle 80 to face one another, in order to calculate a direction of pressure based on the difference between the pressure values detected by the optical fiber sensors 301 facing each other.

FIG. 4 is a schematic diagram of an operation principle of pressure detection by the optical fiber sensor 301. The right side of the diagram indicates the insertion direction. The optical fiber sensor 301 includes a core 401 that propagates light, and a cladding 402 that covers the core 401. The core 401 is arranged from the tip end at regular intervals d.

The operation principle to detect pressure by the optical fiber sensor 301 will be described below. Light having a broad wavelength (broadband incident light) enters from a light source (not illustrated) arranged at the rear end side of the optical fiber sensor 301. In the case where the optical fiber sensor 301 is bent, the intervals between the cores 401 are changed from regular intervals to interval Δd. The broadband incident light returns to the light source side as a reflective light (specific wavelength reflective light) in which a wavelength of a specific band is emphasized proportional to the interval Δd of the cores 401. The interval Δd is inversely proportional to externally applied pressure F, and accordingly, a pressure value and a direction of pressure can be detected from the specific wavelength reflective light.

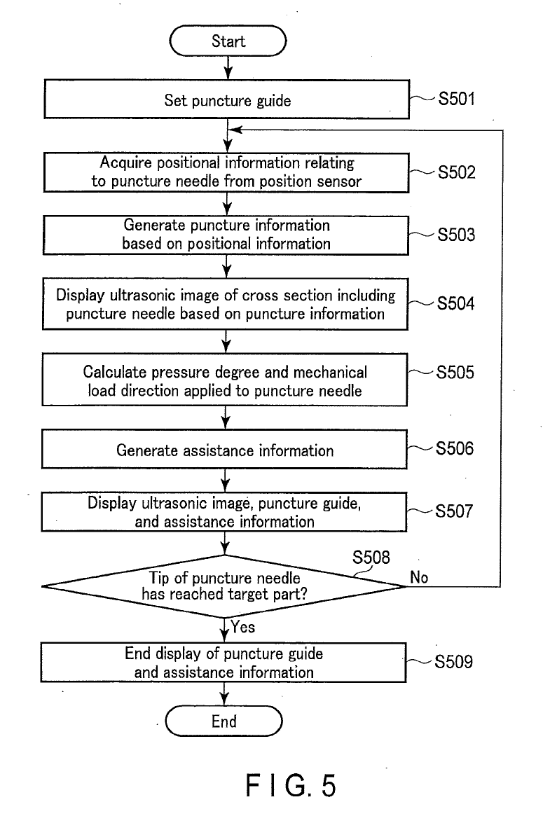

The operation of the ultrasonic diagnostic apparatus 1 according to the first embodiment will be explained with reference to the flowchart of FIG. 5. It is assumed that the ultrasonic image and the puncture guide are displayed in advance.

In step S501, the control circuitry 22, by activating the puncture information generation function 102, sets a puncture guide relative to a target part included in the ultrasonic image. An example method of generating a puncture guide will be described. However, the general method of calculating an expected path of a puncture needle may be applied. The control circuitry 22, by activating the puncture information generation function 102, sets a center of the target part as an end of the puncture guide. Next, the control circuitry 22, by activating the puncture information generation function 102, determines a straight line extending from the end of the puncture guide and falling within a range of a puncture angle of the puncture needle (within a range of an angle set by the puncture adaptor 81) as the puncture guide.

In step S502, the control circuitry 22, by activating the position information acquisition function 101, acquires positional information relating to a position and a direction of the puncture needle 80 from the position sensor 31 attached to the puncture needle 80.

In step S503, the control circuitry 22, by activating the puncture information generation function 102, generates puncture information of the puncture needle 80 based on the positional information.

In step S504, the control circuitry 22, by activating the display control function 104, displays the ultrasonic image of a cross section including the puncture needle based on the puncture information.

In step S505, the control circuitry 22, by activating the puncture information generation function 102, calculates a degree of pressure applied to the puncture needle 80 and a direction of the mechanical load applied to the puncture needle 80 based on pressure information acquired from the pressure sensor provided in the puncture needle 80.

In step S506, the control circuitry 22, by activating the assistance information generation function 103, generates assistance information based on the degree of pressure applied to the puncture needle 80 and the direction of the mechanical load applied to the puncture needle 80 calculated in step S505. The operator can operate the puncture needle 80 along the puncture guide by referring to the assistance information.

In step S507, the control circuitry 22, by activating the display control function 104, displays the ultrasonic image, the puncture guide, and the assistance information.

In step S508, the control circuitry 22, by activating the puncture information generation function 102, determines whether or not a tip of the puncture needle 80 reaches the target part. The determination may be performed, for example, by comparing the positional information (for example, coordinates) of the tip end of the puncture needle with coordinates indicating an area of the target part, and determining whether or not the coordinates of the tip end of the puncture needle 80 is included in the area of the target part. If the tip end of the puncture needle 80 reaches the target part, the processing proceeds to step S509. If the tip end of the puncture needle 80 does not reach the target part, the processing returns to step S502, and repeats steps S502 to S508.

In step S509, the control circuitry 22, by activating the display control function 104, ends display of the puncture guide and the assistance information.

From step S502 to step S508, real time processing is desired, for example, the processing is sequentially performed every one-second. The operation of the ultrasonic diagnostic apparatus 1 according to the present embodiment ends by the above processing.

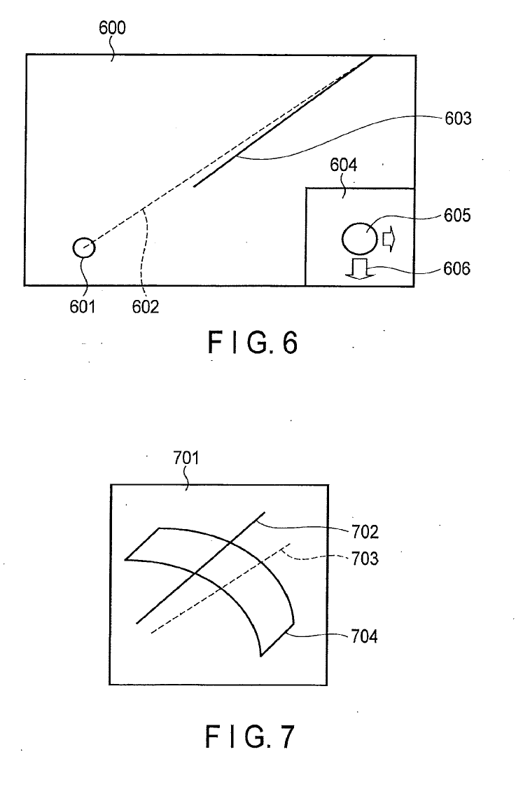

Next, the first display example of the assistance information according to the first embodiment will be described with reference to FIG. 6.

FIG. 6 illustrates a display example of an ultrasonic image 600 generated based on transmission/reception of ultrasonic waves by using the ultrasonic probe 70. A tumor 601 which is a target part to which the puncture needle 80 is inserted is displayed on a screen. A puncture guide 602 to guide a puncture needle to reach the tumor 601 is indicated by a broken line. An image indicating a puncture needle (hereinafter referred to as a puncture needle image 603) is indicated in a solid line. FIG. 6 shows that the puncture needle image 603 is deviated from the puncture guide 602.

An axial image 605 of the puncture needle 80 viewed from the rear side of the puncture needle 80 and an arrow 606 indicating the degree of pressure applied to the puncture needle 80 and the direction of the mechanical load applied to the puncture needle 80 are displayed as assistance information 604. The axial image 605 is a cross sectional view of a tip of the puncture needle 80 in which the pressure sensor 82 is provided (for example, at a point 3 cm from the end). The degree of pressure is proportional to the pressure value, and is represented as a thickness of the arrow 606. For example, the thickness of the arrow 606 increases as the degree of pressure increases. However, a numerical value of the pressure value may be indicated along with the arrow 606.

In FIG. 6, the two arrows 606 represent the two directions of pressures for which the difference between the values of a pair of pressure sensors 82 facing each other among four pressure sensors 82 has been calculated. It may be possible to indicate the degree of pressure and the direction of pressure with one arrow by calculating the difference between the pressure values between all the pressure sensors 82 in consideration of the arrangement of each pressure sensor. That is, in the example of FIG. 6, an arrow directed approximately to the right lower direction of the axial image 605 may be displayed.

The operator can recognize that the mechanical load is applied to the puncture needle 80 in the right lower direction from the operator side by referring to the assistance information 604 as shown in FIG. 6. The operator therefore can easily realize that the puncture needle 80 tends to be deviated from the puncture guide 602 in the direction where the mechanical load is applied if the puncture needle 80 is inserted as-is. The operator can easily realize how to correct the insertion direction based on the assistance information 604. The correction of the insertion direction may be performed by withdrawing and inserting the puncture needle 80 again, or turning the tip, for example, by rotating the puncture needle 80 by 180 degrees.

The assistance information may include information to correct the deviation of the puncture needle from the puncture guide. The second display example of the assistance information will be described with reference to FIG. 7.

FIG. 7 illustrates an extract of the assistance information displayed along with the ultrasonic image.

A puncture angle 702 of the puncture needle, a correction angle 703 of the puncture needle, and a puncture adaptor image 704 are displayed as assistant information 701. The puncture angle 702 represents an actual insertion angle of the puncture needle 80 and a path of the puncture needle 80, and is indicated by a solid line. The puncture angle 702 is an angle along the puncture guide, in the initial stage. The puncture angle 702 may be an angle set by the puncture adaptor 81, for example. A numerical value of an angle may be added along the solid line, or may be indicated instead of the solid line. A correction angle 703 represents an angle of the puncture needle 80 to correct the puncture needle 80 deviated from the puncture guide, and is indicated by a broken line. The correction angle 703 may be indicated by a numerical value of an angle added along the broken line, or may be indicated only by a numerical value of the angle.

The operator can easily realize the angle of inserting the puncture needle 80 to the living body in order to reach the target part by referring to the assistant information 701 as shown in FIG. 7. Accordingly, the angle of the puncture needle 80 can be adjusted in accordance with the correction angle 703, and the puncture needle 80 can be re-inserted at an appropriate angle.

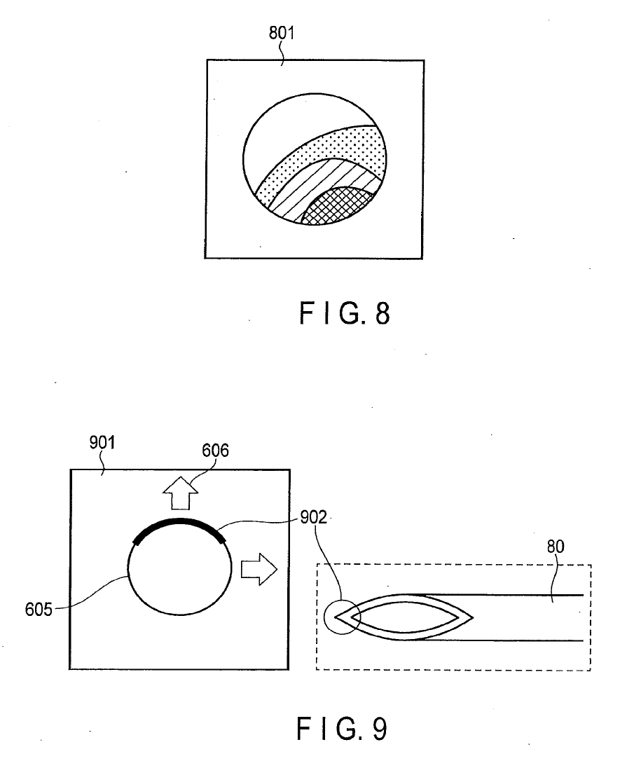

In addition, as shown in FIG. 8, the degree of pressure and the direction of pressure applied to the puncture needle 80 may be represented as contour lines or a color map, which is the third display example of the assistance information. Assistance information 801 represents the values of the pressure sensors 82 attached to the puncture needle as they are. By representing the distribution of pressure values as contour lines or a color map as the assistance information 801, the pressure value and the direction of the mechanical load applied to the puncture needle 80 can be instinctively realized similarly to the case where arrows are used as the assistance information. In the example of FIG. 8, it is indicated that the pressure is externally applied to the puncture needle 80 from the right lower direction in the cross section. Accordingly, the operator expects that the puncture needle 80 is going to shift (curve) in the left upper direction.

In addition to the aforementioned display example of the assistance information, information relating to the position of a needle tip end (needle tip end position) of the puncture needle 80 or a blade surface of the puncture needle tip may be displayed. In the present embodiment, the needle tip end is an edge of the puncture needle 80 that is firstly brought into contact with a living body P. A display example in which information on the needle tip end position is added to the assistance information is illustrated in FIG. 9.

The puncture needle 80 is generally formed in a manner that a tip end is canted off to form a blade surface and a sharp tip. The direction of inserting the puncture needle 80 is mostly determined by the orientation of the blade surface. Specifically, while the puncture needle 80 is being inserted into the living body, the puncture needle 80 is going to curve to the direction opposed to the blade surface, i.e., the tip of the puncture needle 80 is warped in many cases.

Accordingly, as shown in FIG. 9, the needle tip end position 902 of the puncture needle is displayed in addition to the arrow 606 as assistance information 901. The needle tip end position 902 of the puncture needle can be detected by associating the needle tip end with a predetermined position of a pressure sensor. The operator can recognize the needle tip end position 902 by referring to the assistance information 901, and can further improve the prediction accuracy of the direction in which the puncture needle is to be deviated from the puncture guide. The needle tip end position 902 is indicated as a solid arc having a predetermined angle range in FIG. 9. However, the needle tip end position 902 may be indicated by a dot or by coloring a portion corresponding to the tip of the puncture needle 80 among a circle representing an axial image 605. That is, the needle tip end position 902 may be indicated in any way if the needle tip end position is specified.

The puncture needle 80 may be displayed as a three-dimensional model so that the operator can recognize the blade surface more easily.

In the case where the needle tip end position 902 is displayed as the assistance information 901, an arrow 606 may indicate the direction to which the puncture needle 80 is to be moved to correct the deviation from the puncture guide instead of displaying the direction of pressure applied to the puncture needle 80.

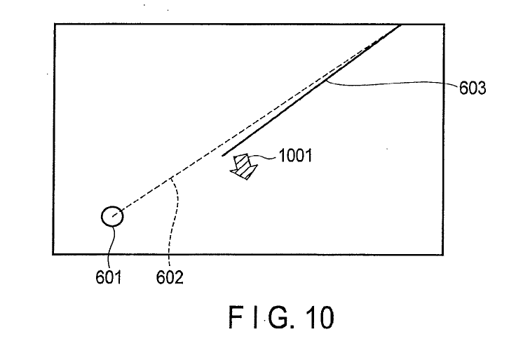

As shown in FIG. 10, assistance information is indicated by an arrow 1001 near the tip of the puncture needle image 603, instead of displaying the assistance information in a window different from the ultrasonic image. This is the fourth display example of the assistance information. As stated above, the assistance information may be indicated in any aspect if the degree and the direction of the pressure can be recognized.

According to the aforementioned first embodiment, the degree and the direction of pressure applied to the puncture needle are calculated by the pressure sensors, and the assistance information to assist the operation of the puncture needle is generated. Therefore, a deviation of the puncture needle from the puncture guide can be quantitatively detected, and the operator can easily realize how to correct the deviation from the puncture guide. This configuration can assist accurate puncturing in paracentesis regardless of the puncturing skill of the operator. In particular, this embodiment has advantages in the case of requiring accuracy of puncturing, for example, in the case where a target part is tiny such as early-stage cancer.

### Second Embodiment

In the case where deviation of the puncture needle from the puncture guide is corrected based on the assistance information according to the first embodiment, pressure from the operation is applied to the puncture needle. Accordingly, if the operator is correcting the deviation of the puncture needle at a time when the assistance information is generated, there is a possibility that incorrect assistance information may be generated based on pressure other than the pressure applied due to insertion of the puncture needle into the living body. In this case, the operator may operate the puncture needle incorrectly, based on the incorrect assistance information. There may be a case where real-time assistance information may be annoying for the operator. In the second embodiment, the assistance information can be switched between an on-state and an off-state at predetermined intervals or manually so that assistance information can be suitably generated.

The ultrasonic diagnostic apparatus according to the second embodiment performs the operations similar to the ultrasonic diagnostic apparatus of the first embodiment except the operation of the display control function 104.

The control circuitry 22, by activating the display control function 104, switches on and off states of the assistance information display in accordance with the degree of insertion of the puncture needle 80 into a living body. The display of assistance information may be switched as indicated below. For example, in the initial setting, display of assistance information is in the on-state, the display is switched to the off-state for a predetermined time (for example, for three seconds) if the puncture needle is inserted a predetermined distance (for example, 1 cm) into a living body, and after a predetermined time has elapsed, the display is switched to the on-state. On the other hand, the display of assistance information is in the off-state in the initial setting, if the puncture needle inserted a predetermined distance into a living body, the display is switched to the on-state for a predetermined time, and after a predetermined time has elapsed, the display is switched to the off-state. The operator may correct the insertion direction of the puncture needle while the display of assistance information is in the off-state.

Alternatively, the control circuitry 22, by activating the display control function 104, may switch on/off states of the assistance information in response to the operator's instruction.

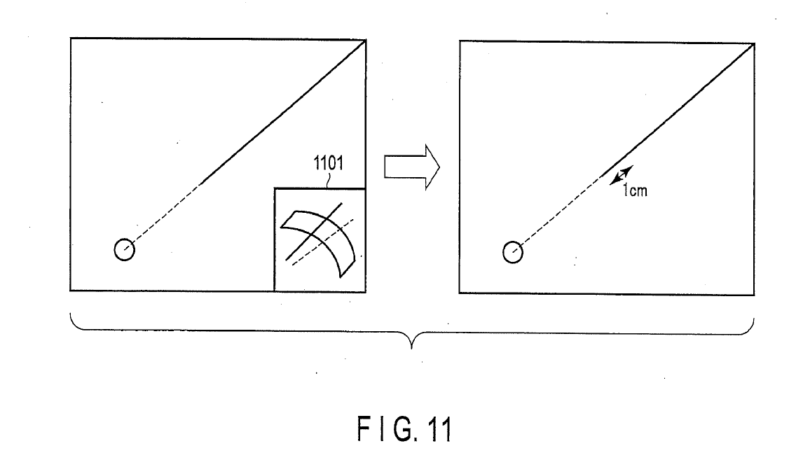

An example of display control of the assistance information by the display control function 104 will be explained with reference to FIG. 11.

FIG. 11 illustrates the case where display of assistance information is in the on-state in the initial setting. As shown in the left drawing, if the puncture needle is inserted 1 cm in the state where assistance information 1101 is displayed, the display of the assistance information 1101 comes to be in the off-state. The display control function 104 may control the display of the assistance information 1101 to be downsized or to be translucent (increase transparency), instead of completely turning off the display of the assistance information 1101. That is, the assistance information 1101 may be displayed in any form different from normally displayed assistance information so that the assistance information 1101 can be recognized as the information that the operator should not refer to.

According to the aforementioned second embodiment, the display of assistance information is switched between on/off states by the display control function so that assistance information generated based on inappropriate information such as pressure applied to the puncture needle from the operator is not displayed, or the form of display is changed by the display control function so that the operator can distinguish the assistance information generated based on inappropriate information. Therefore, updated assistance information can be displayed to the operator at a suitable timing. The operator can reduce the possibility of operating the puncture needle based on incorrect assistance information, and assistance for highly accurate puncturing can be provided.

It is also possible to display an expected insertion line indicating an expected insertion direction of the puncture needle on the ultrasonic image explained in the aforementioned embodiments.

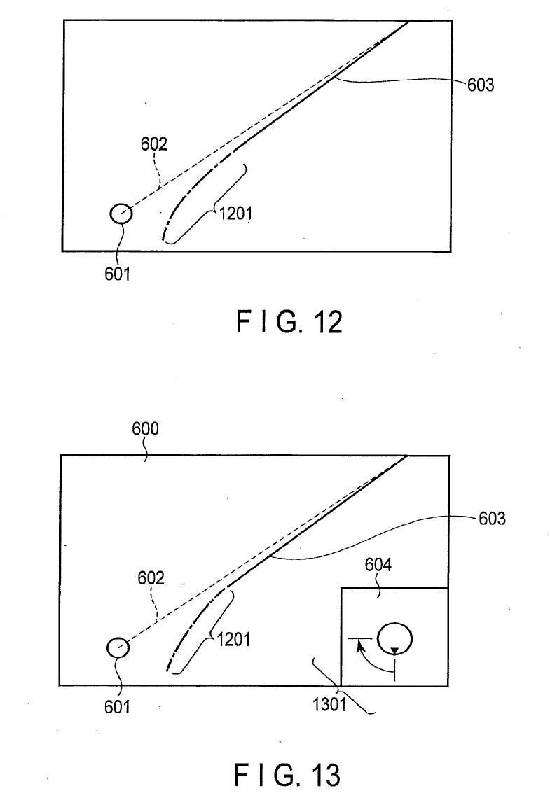

A display example of an expected insertion line will be described with reference to FIG. 12.

In the example shown in FIG. 12, the puncture guide 602 is displayed by a broken line, and an expected insertion line 1201 of the puncture needle is displayed by an alternating long and short dashed line on the ultrasonic image. The expected insertion line 1201 is generated as indicated below.

For example, a pressure value applied to the puncture needle and a distortion amount and a distortion direction of the puncture needle (deviation from a geometric extension of the puncture needle without the distortion) are stored as being associated with each other in the internal storage 17. The control circuitry 22, by activating the assistance information generation function 103, generates a path of the expected insertion line 1201 in accordance with the distortion direction and the distortion amount corresponding to the measured pressure value.

By displaying the expected insertion line 1201, the operator can easily realize in what degree the puncture needle curves or in what degree the puncture needle is deviated from the tumor 601 or the puncture guide 602 if the puncture needle is inserted as-is. In the case where the puncture guide 602 and the expected insertion line 1201 are displayed at the same time, this information may be differentiated based on at least one of a type of line, a thickness, or a color. Only the expected insertion line 1201 may be displayed without displaying the puncture guide 602.

It is also possible to display correction information indicating in what degree the needle tip should be rotated to adjust the path of the puncture needle to reach the tumor 601 as assistance information.

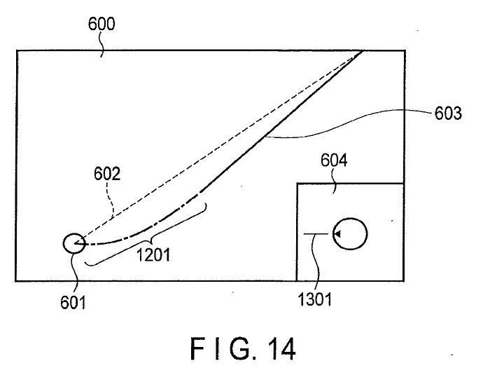

An example of a case where correction information is displayed as assistance information will be explained with reference to FIGS. 13 and 14.

FIG. 13 illustrates an example case where the assistance information 604, the puncture guide 602, and the expected insertion line 1201 are displayed on the ultrasonic image 600. In FIG. 13, the expected insertion line 1201 shows that the insertion direction of the puncture needle is expected to be away from the tumor 601.

Correction information 1301 indicating a correction required for the tip of the puncture needle to reach the tumor 601 is displayed as the assistance information 604. The correction information 1301 is, for example, information indicating to what degree the puncture needle is required to be rotated (the amount of rotation) in which direction from the current state. In the example of FIG. 13, the needle tip end position is indicated by a triangle mark, and the rotation amount is indicated by using two bars. FIG. 13 shows that the correction information 1301 indicates that the puncture needle needs to be rotated clockwise by 45 degrees to reach the tumor 601. By this display, the operator can easily realize how to correct the puncture needle instinctively. In the case where it is difficult for the puncture needle to reach the tumor 601 only by the rotation correction of the puncture needle, information used for correcting an insertion distance of the puncture needle or information indicating that it is necessary to withdraw the puncture needle and reset a puncture initial position is displayed as correction information. The correction information 1301 may be displayed in any way if the operator can understand the indication of the correction information 1301.

The correction information 1301 may be obtained, for example, by calculating the rotation direction and the rotation amount of the tip of the puncture needle based on the deviation amount of the expected insertion line 1201 from the extension of the puncture guide 602 and the position of the tumor 601. The rotation amount may be represented as a numeric value such as “45°” on a screen, or expressed as beeps which are set to be sounded in shorter intervals as the rotation amount becomes closer to the calculated rotation amount.

The result where the puncture needle is rotated based on the correction information 1301 is illustrated in FIG. 14.

As shown in FIG. 14, as a result where the puncture needle is rotated by the amount indicated by the correction information 1301, the expected insertion line 1201 is updated to be directed to the tumor 601.

While the operator is rotating the puncture needle, the expected insertion line 1201 may be updated in real-time in accordance with the rotated amount. To update the expected insertion line 1201, the control circuitry 22, by activating the assistance information generation function 103, three-dimensionally calculates the expected insertion line 1201 in accordance with the rotation of the puncture needle so that the expected insertion line 1201 is directed to the direction of the needle tip (the needle tip is warped from the extension of the puncture needle), and projects the calculated expected insertion line 1201 on the plane where the ultrasonic image is displayed.

The functions described in connection with the above embodiments may be implemented, for example, by installing a program for executing the processing in a computer, such as a work station, etc., and expanding the program in a memory. The program that causes the computer to execute the processing can be stored and distributed by means of a storage medium, such as a magnetic disk (a hard disk, etc.), an optical disk (CD-ROM, DVD, Blu-ray® Disc, etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising a processor configured to:
generate assistance information to assist an operation of a puncture needle based on pressure information acquired from a pressure sensor attached to the puncture needle; and
display on a display the assistance information and an ultrasonic image which includes at least an image of a tip of the puncture needle, the ultrasonic image being acquired by transmission and reception of ultrasonic waves by using an ultrasonic probe.

2. The apparatus according to claim 1, wherein the assistance information includes information relating to a degree of pressure applied to the puncture needle and a direction of a mechanical load applied to the puncture needle.

3. The apparatus according to claim 1, wherein the processor is further configured to display on the display a puncture guide indicating an expected insertion path of the puncture needle.

4. The apparatus according to claim 3, wherein the assistance information includes a puncture angle of the puncture needle and a correction angle to correct deviation from the puncture guide.

5. The apparatus according to claim 1, wherein the processor generates puncture information that includes at least a position of the tip of the puncture needle and a insertion direction of the puncture needle, by using positional information regarding the puncture needle acquired from a position sensor, and
wherein the ultrasonic image is a cross sectional view including the puncture needle and is generated based on the puncture information.

6. The apparatus according to claim 1, wherein the assistance information includes information regarding a needle tip end position of the puncture needle.

7. The apparatus according to claim 1, wherein the pressure sensor is formed by one of an optical fiber, a metallic thin film, and a semiconductor.

8. The apparatus according to claim 1, wherein the processor controls at least one of turning on and off of display of the assistance information and a form of display of the assistance information, in accordance with a degree of insertion of the puncture needle.

9. The apparatus according to claim 1, wherein the processor displays on the display an expected insertion line indicating an expected insertion path of the puncture needle.

10. The apparatus according to claim 1, wherein the assistance information includes correction information to correct a path of the puncture needle.

11. An ultrasonic diagnostic method comprising:
generating assistance information to assist an operation of a puncture needle based on pressure information acquired from a pressure sensor attached to the puncture needle; and
displaying on a display the assistance information and an ultrasonic image which includes at least an image of a tip of the puncture needle, the ultrasonic image being acquired by transmission and reception of ultrasonic waves by using an ultrasonic probe.
